(19) United States
(12) Patent Application Publication
(10) Pub. No.: US 2018/0005290 A1
(43) Pub. Date: Jan. 4, 2018

(54) SYSTEM AND METHOD FOR PROVIDING CUSTOMIZED USER EXPERIENCE WITH ABSORBENT ARTICLES

(71) Applicant: The Procter & Gamble Company, Cincinnati, OH (US)

(72) Inventors: John Gregory Warzala, Cincinnati, OH (US); Mattias Schmidt, Idstein (DE)

(21) Appl. No.: 15/488,834

(22) Filed: Apr. 17, 2017

Related U.S. Application Data
(60) Provisional application No. 62/356,847, filed on Jun. 30, 2016.

(51) Int. Cl. G06Q 30/06; A61F 13/15

(52) U.S. Cl. CPC G06Q 30/0621; A61F 13/15203

(57) ABSTRACT

Included are embodiments for a computer based system and method for providing consumers with mass customized personal hygiene absorbent articles. Some embodiments include registering a user for participation (or allowing them to participate as a guest user) in an online customizing and purchasing system, in which one or more absorbent articles, e.g. diapers, are customized in response to input about a child intended to be the wearer of such absorbent articles, the input being provided by a user, and a user being provided with the opportunity to purchase such customized absorbent articles.

### CROSS REFERENCE TO RELATED APPLICATION

This application claims the benefit of U.S. Provisional Application No. 62/356,847, filed Jun. 30, 2016, the substance of which is incorporated herein by reference.

### FIELD OF THE INVENTION

The present application relates generally to systems and methods for allowing users to experience a customized experience in selecting personal hygiene absorbent articles, such as diapers or pants, and adult incontinence products (for example, liners, pads, briefs, and pants).

### BACKGROUND OF THE INVENTION

Various industries provide consumers with the ability to obtain customized products. They range from highly technical products, such as computers, where a consumer may “create-their-own” product by selecting the specification of the computer's individual components, such as quantity of memory, speed of microprocessor, size and resolution of screen, selection of input/output ports, etc., to purely aesthetic customization, such as chocolate candies, where a consumer may have a personalized saying or set of initials imprinted on the candies.

Recently, it has been proposed to provide consumers with mass customization in the consumer goods sectors, including mass customization and personalization for personal hygiene absorbent articles (see U.S. Ser. No. 15/163,757 (Schmidt, et al.)). Increasingly, consumers wish to be able to order customized products using computer interfaces. Thus, there is a need to provide a computer based system and method for providing consumers with mass customized personal hygiene absorbent articles. Personal hygiene articles are disclosed generally in WO2012/170778 (Rosati, et al.).

### SUMMARY OF THE INVENTION

Included are embodiments for a computer based system and method for providing consumers with mass customized personal hygiene absorbent articles. Some embodiments include registering a user for participation (or allowing them to participate as a guest user) in an online customizing and purchasing system, in which one or more absorbent articles, e.g. diapers, are customized in response to input about a child intended to be the wearer of such absorbent articles, the input being provided by a user, and a user being provided with the opportunity to purchase such customized absorbent articles.

Also included are embodiments of a computing device. Some embodiments of the computing device include a memory component that stores a program to carry out the system and methods described herein.

Also included are embodiments of a non-transitory computer-readable medium. Some embodiments of the non-transitory computer-readable medium include a program that causes a computing device to carry out the system and methods described herein.

### BRIEF DESCRIPTION OF THE DRAWINGS

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate various embodiments described herein, and together with the description serve to explain the principles and operations of the claimed subject matter.

FIG. 1 depicts a computing environment for providing a method for providing consumers with mass customized personal hygiene absorbent articles, according to embodiments disclosed herein.

### DETAILED DESCRIPTION OF THE INVENTION

Embodiments disclosed herein include systems and methods for providing consumers with mass customized personal hygiene absorbent articles.

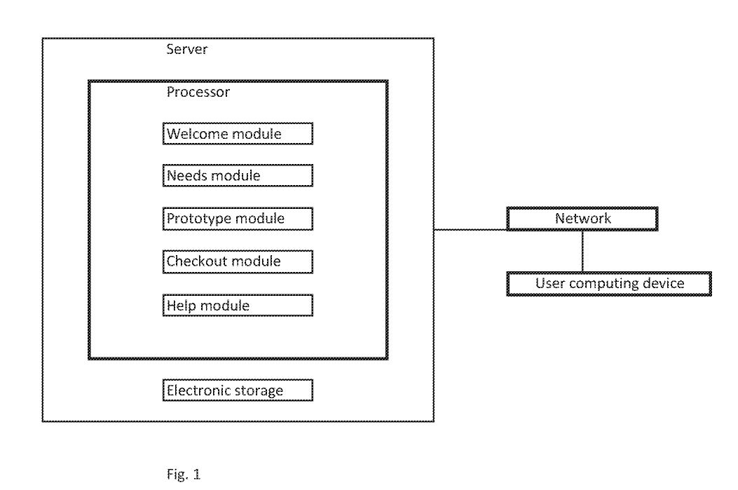

Referring now to the drawings, FIG. 1 depicts a computing environment for providing customized advice messaging, according to embodiments disclosed herein. As illustrated, a network may be coupled to a user computing device. The network may include a wide area network and/or a local area network and thus may be wired and/or wireless. The user computing device may include any portable and/or non-portable computing devices, such as personal computers, laptop computers, personal digital assistants (PDAs), tablet computers, mobile phones including those commonly referred to as smart phones, smart watches, and smart eyeglasses, etc. In some embodiments, the user computing device may be configured to send and/or receive data related to the method for providing consumers with mass customized personal hygiene absorbent articles.

The user computing device may include a memory component that stores user registration logic (among other pieces of logic). The user registration logic may include software, hardware, and/or firmware to provide user interfaces for registering a user and/or allowing the user to interface with the system.

Not shown is a remote computing device for providing consumers with mass customized personal hygiene absorbent articles, according to embodiments disclosed herein. The remote computing device includes a processor, input/output hardware, network interface hardware, a data storage component, and the memory component. The memory component may be configured as volatile and/or nonvolatile memory and as such, may include random access memory (including SRAM, DRAM, and/or other types of RAM), flash memory, secure digital (SD) memory, registers, compact discs (CD), digital versatile discs (DVD), and/or other types of non-transitory computer-readable mediums. Depending on the particular embodiment, these non-transitory computer-readable mediums may reside within the remote computing device and/or external to the remote computing device.

Additionally, the memory component may store operating logic, the user registration logic, and any other logic. A local communication interface may be implemented as a bus or other communication interface to facilitate communication among the components of the remote computing device.

The processor may include any processing component operable to receive and execute instructions (such as from the data storage component and/or the memory component). The input/output hardware may include and/or be configured to interface with a monitor, positioning system, keyboard, touch screen, mouse, printer, image capture device (such as a still image camera, a video camera, a 2-dimensional camera, 3-dimensional camera, etc.), microphone, speaker, gyroscope, compass, and/or other device for receiving, sending, and/or presenting data. The network interface hardware may include and/or be configured for communicating with any wired or wireless networking hardware, including an antenna, a modem, LAN port, wireless fidelity (Wi-Fi) card, WiMax card, mobile communications hardware, and/or other hardware for communicating with other networks and/or devices. From this connection, communication may be facilitated between the remote computing device and other computing devices.

The operating logic may include an operating system and/or other software for managing components of the remote computing device. Similarly, as discussed above, the user registration logic may reside in the memory component and may be configured to cause the processor to provide user interfaces and register a user with the customized advice messaging service. Similarly, the user question logic may be utilized to facilitate the answering of questions and providing of pieces of advice. Other functionality is also included and described below. It should be understood that the components described above are merely exemplary and are not intended to limit the scope of this disclosure.

A “welcome” module may be provided, which may include a user interface that may be provided to a user for accessing the system, according to embodiments disclosed herein. The welcome module may communicate with the user computing device to provide a user interface which may be utilized for registering a user with the customized advice messing service. Depending on the particular embodiment, the user may be the parent of an unborn child who is expected to wear absorbent articles or recently born child who is currently wearing absorbent articles. In some embodiments, the user may be a friend, relative and/or caregiver of the child. In some embodiments, the user may log into a website, an intranet site, and/or other portal for providing the user interface. In other embodiments, user may engage the user interface at a retail store, a retail kiosk, or other place of sale. The welcome module may provide a previous user option that allows a user to indicate whether they are an existing user of the system or a new user. An existing user may be prompted to enter a username and password or similar entry gating protocol. A new user may be prompted to create a profile of themselves and one or more children. Such a user profile may include personal information, like name, shipping address, credit card info, email address, etc. Care should be taken to observe relevant privacy concerns. Such a child profile may include basic information about a child, at least sufficient to associate that child profile with the user's profile. The welcome module may prompt the user to create or update information about a one or a plurality of children and associate this child profiles with the user's profile. For example, a user may be prompted to indicate the child's name (or nickname), age, gender. Again, care should be taken to observe relevant privacy concerns. An existing user may be prompted to select which child they wish to direct a given engagement of the system towards, and also be prompted to update any information in either their profile or any one of child profiles associated with their user profile.

A “needs” module may allow a user to indicate certain information about a child related to the child's needs (and perhaps the user's wants) in an absorbent article (for example, diaper or pant) to be worn by the child. For example, a user may be prompted to indicate, for the selected child, one or more characteristics of: a diaper/pant size (perhaps suggesting sizes according to the child's weight); motor activity level (e.g. laying, crawling, walking); texture of materials for comfort (e.g. quilted and plush, silky and smooth, gentle and breathable); skin type (e.g. pure, sensitive, prone to rash); and style (e.g. trendy, classic, creative). Other characteristics may be included. In an adult incontinence context, a user may be prompted to indicate one or more of the following exemplary characteristics and/or desires: weight, body mass index, panty size, body shape (for example, rectangle or cylindrical, hourglass, pear, apple), texture of materials, desired panty type (for example, hipster, high leg, brief, brief high waist, high waist and leg), desired color (for example, white, black, nude, mauve), underwear design features (for example, polka dots, lace), inclusion of a scent or be scent free, inclusion of odor control or not, inclusion of skin enhancing ingredient/lotion or not, absorbency level (for example, moderate, maximum, maximum plus), and discretion or reassurance. Additional user characteristics and/or desires that can be communicated to a needs module are described in co-pending U.S. patent application Ser. No. 14/698,924, the relevant portions of which are incorporated herein by reference.

A “prototype” module may allow a user to see (on screen) a mock-up of diaper/pant prepared by the system based on the input from the needs module. For example, the system may be provided with one or more algorithms to select certain technical diaper/pant components in response to the input that the user provided in the needs module. For example, in response to an indication that the child's activity level is walking, the system may suggest a diaper/pant with more elasticity in the waistband and leg cuffs than if the child's activity level is laying. In another example, in response to an indication that the child's texture of materials for comfort is quilted and plush, the system may suggest a diaper/pant with a higher basis weight, less apertured nonwoven than if the child's comfort material was breathable. In yet another example, in response to an indication that the skin type is prone to rash, the system may suggest a diaper/pant that is free of scent producing ingredients than if the child's skin type is pure. In an adult incontinence context, in response to body weight, body mass index, and/or body shape, the system may suggest an adult incontinence brief/pant that has a particular leg cut (low, medium, high) and/or waist level (low, high). By way of example only, the system may suggest an incontinence brief/pant that has more coverage (low to medium leg cut and high waist) to users with higher weight and/or body mass index (for example, greater than or equal to 30), and may suggest an incontinence brief/pant that has a higher leg cut (medium to high) and low waist to users with relatively lower weight and/or body mass index (for example, less than or equal to 25). Once an adult incontinence brief/pant with a particular leg cut and/or waist level is recommended to a user, the user can then choose other aspects of the brief/pant; for example, color, scent, lotion, odor control, material, and absorbency level.

It is contemplated that the prototype module may provide the user with information that helps correlate the system's suggestions to the user's input from the needs module. For example, if the user indicated that the child's activity level was walking, the user may be informed that the suggested diaper/pant has a stretchy garment-style waistband and leg cuffs, designed for a comfortable fit while the child is in frequent motion. In another example, if the user indicated that the child's overnight wetting was high, the user may be informed that the suggested diaper/pant has an increased amount of superabsorbent material with channel-forming areas, to help absorb more urine and move it away from the child's skin overnight.

The prototype module may further provide the user the opportunity to select the diaper/pant or adult incontinence product suggested by the system or to modify it in any way. The system may prompt the user to indicate whether they wish to select the diaper/pant or adult incontinence product as is, or modify any one or more of the components of the suggested diaper/pant or adult incontinence product. In response to the user indicating that they wish to modify a component, the system may return the user to the portion of the needs module where the input what was collected that resulted in the particular component being suggested. For example, if the selected component for modification is that the diaper/pant or adult incontinence product is scent-free, the system may prompt the user to edit their indication of whether the child's or their skin is pure, sensitive, or prone to rash. The user can then change their selection from prone to rash to sensitive. Additionally, the system may simply allow the user to edit the final component without having to re-enter or edit information in the needs module. The prototype module may also provide the user the option to save their customized settings, optionally under a unique identifier like, e.g. “Sophie's crawling diaper”, or “Rori's overnight sleep diaper”. The system may associate the unique diaper/pant or adult incontinence product with the child's or their profile to enable the user to select it again during a future visit to the system.

The prototype module may also allow a user to see images of customized diapers/pants or adult incontinence products created by other users, “trending” diapers/pants or adult incontinence product designs, and/or other suggested diapers/pants or adult incontinence products. Care should be taken to observe relevant privacy concerns. Users may be allowed to modify the diaper/pant or adult incontinence product suggested by the system in view of these other customized or suggested diapers/pants or adult incontinence products, or may select them in place or in addition to the diaper/pant or adult incontinence product suggested by the system.

A “checkout” module may be provided to allow a user to purchase the diaper/pant or adult incontinence product that they selected in the prototype module or any diaper/pant or adult incontinence product that they may have saved in a child's or their profile. Users may be prompted to purchase various quantities of the diapers/pants or adult incontinence products that they have selected, to provide payment information, to provide delivery information, and like information typically associated with on-line purchasing. Users may also be prompt to enter, or may be offered information relating to coupons, loyalty discounts, shipping discounts, etc.

A “help” module may be associated with any other module to provide a user with assistance. Such assistance may be related to technical issues, payments, any of the inputs being provided by the user, etc. Such module may also be provided with Frequently Asked Questions (and their answers), telephone contact information for recorded or live assistance from another, live chat contact information for live assistance from another, a Community questions/answers section, etc.

A “user relationship” module may be provided to communicate with the user indirectly from the user's use of the system online. The user relationship module may send messages to the user who may receive them on any one or more of the computing devices they provide contact information for in the user's profile. Such messages may be to provide information about a purchase made by the user, e.g. a receipt, shipping details, etc., about a purchase made by another user as a gift for a child associated with the user's profile, about new product offerings available through use of the system, about discounts, sales, and other retail offers and promotions, to remind the user that it may be time to order more diapers/pants or adult incontinence products, and/or otherwise provide helpful advice to the user.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be understood to those skilled in the art that various other changes and modifications can be made without departing from the spirit and scope of the invention. It is therefore intended to cover in the appended claims all such changes and modifications that are within the scope of this invention.

What is claimed is:

1. A method for providing consumers with mass customized personal hygiene absorbent articles, comprising:
registering a user to customize personal hygiene absorbent articles, wherein registering the user comprises receiving and storing user information;
receiving, from the user input relating to a child or adult intended to wear customized absorbent articles, wherein the input relates to at least one characteristic of the child or adult that influences the functional features of the customized absorbent article;
utilizing the information obtained in the prior step to select absorbent article components that functionally relate to the characteristics of the prior step;
combining the absorbent article components selected in the prior step into a prototype absorbent article and providing an image and/or description of such article to the user;
receiving from the user input either selecting the prototype absorbent article of the prior step or expressing a the choice to modify the prototype absorbent article;
in response to the selection of a final prototype absorbent article, providing the user with an option to purchase said final prototype absorbent article.

2. The method of claim 1, wherein the absorbent article is an adult incontinence brief or pant.

3. The method of claim 2, wherein the input comprises weight or body mass index.

4. The method of claim 3, wherein when the body mass index is greater than or equal to 30, the prototype absorbent article comprises a low to medium leg cut among a choice of low, medium, or high leg cut.

5. The method of claim 3, wherein when the body mass index is greater than or equal to 30, the prototype absorbent article comprises a high weight level among a choice of low or high waist level.

6. The method of claim 3, wherein when the body mass index is less than or equal to 25, the prototype absorbent article comprises a medium to high leg cut among a choice of low, medium, or high leg cut.

7. The method of claim 3, wherein when the body mass index is less than or equal to 25, the prototype absorbent article comprises a low weight level among a choice of low or high waist level.

8. The method of claim 2, wherein the input comprises a body shape selected from a group comprising at least one of rectangle, cylindrical, hourglass, pear, and apple.

9. The method of claim 8, wherein when the body shape is pear or apple, the prototype absorbent article comprises a low to medium leg cut among a choice of low, medium, or high leg cut.

10. The method of claim 8, wherein when the body shape is pear or apple, the prototype absorbent article comprises a high weight level among a choice of low or high waist level.

11. The method of claim 2, wherein the prototype absorbent article comprises a recommended waist level among low and high, and wherein the user is presented with an opportunity to modify the prototype absorbent article by selecting a color of the final prototype absorbent article.

12. The method of claim 2, wherein the prototype absorbent article comprises a recommended waist level among low and high, and wherein the user is presented with an opportunity to modify the prototype absorbent article by selecting the final prototype absorbent article to comprise a scent or to be scent-free.

13. A computing device for customized advice messaging, comprising:
a memory component that stores a program for performing at least the following:
registering a user to customize personal hygiene absorbent articles, wherein registering the user comprises receiving and storing user information;
receiving, from the user input relating to a child or adult intended to wear customized absorbent articles, wherein the input relates to at least one characteristic of the child or adult that influences the functional features of the customized absorbent article;
utilizing the information obtained in the prior step to select absorbent article components that functionally relate to the characteristics of the prior step;
combining the absorbent article components selected in the prior step into a prototype absorbent article and providing an image and/or description of such article to the user;
receiving from the user input either selecting the prototype absorbent article of the prior step or expressing a the choice to modify the prototype absorbent article;
in response to the selection of a final prototype absorbent article, providing the user with an option to purchase said final prototype absorbent article.

14. A non-transitory computer-readable medium for customized advice messaging that stores a program causes a computing device to perform the following:
registering a user to customize personal hygiene absorbent articles, wherein registering the user comprises receiving and storing user information;
receiving, from the user input relating to a child or adult intended to wear customized absorbent articles, wherein the input relates to at least one characteristic of the child or adult that influences the functional features of the customized absorbent article;
utilizing the information obtained in the prior step to select absorbent article components that functionally relate to the characteristics of the prior step;
combining the absorbent article components selected in the prior step into a prototype absorbent article and providing an image and/or description of such article to the user;
receiving from the user input either selecting the prototype absorbent article of the prior step or expressing a the choice to modify the prototype absorbent article;
in response to the selection of a final prototype absorbent article, providing the user with an option to purchase said final prototype absorbent article.
